(19) United States
(12) Patent Application Publication
(10) Pub. No.: US 2011/0313290 A1
(43) Pub. Date: Dec. 22, 2011

(54) CATHETER, APPARATUS METHOD AND COMPUTER PROGRAM FOR APPLYING ENERGY TO AN OBJECT

(75) Inventors: Johannes Wilhelmus Weekamp, Eindhoven (NL); Szabolcs Deladi, Eindhoven (NL); Maya Ella Barley, Eindhoven (NL); Antonius Johannes Josephus Rademakers, Eindhoven (NL); Jan Frederik Suijver, Eindhoven (NL)

(73) Assignee: KONINKLIJKE PHILIPS ELECTRONICS N.V., EINDHOVEN (NL)

(21) Appl. No.: 13/203,732

(22) PCT Filed: Mar. 2, 2010
(86) PCT No.: PCT/IB2010/050886

§ 371 Date: Sep. 1, 2011

(30) Foreign Application Priority Data
Mar. 9, 2009 (EP) 09154615.0

(51) Int. Cl. A61B 8/00; A61B 18/18

(52) U.S. Cl. 600/439; 606/41

(57) ABSTRACT

The present invention relates to a catheter for applying energy to an object. The catheter (5) has a longitudinal axis (7) and includes an energy applying unit (30) for applying energy to the object and a sensing unit (20) for sensing the object in a sensing direction (23). The sensing unit (20) is adapted to be rotatable with respect to the catheter (5) such that the angle (Î±) between the sensing direction (23) of the sensing unit (20) and the longitudinal axis (7) of the catheter (5) is adjustable. The sensing unit (20) can be adjusted during the application of energy to the object such that the sensing direction (23) of the sensing unit (20) points to the site to which the energy is applied, regardless of the angle that the catheter tip forms with the surface of the object at that site.

### FIELD OF THE INVENTION

The present invention relates to a catheter, an apparatus, a method and a computer program for applying energy to an object.

### BACKGROUND OF THE INVENTION

Catheters and apparatuses for applying energy to an object and sensing the object are, for example, used in the field of interventional treatment of atrial fibrillation. During the interventional procedure cardiac tissue is denaturized by thermal therapy. Radio frequency (RF) energy is applied to the cardiac tissue by an ablation catheter, wherein due to resistive losses in the tissue the myocardium is heated up and the heated muscle cells in the cardiac tissue die off and lose their biological function. Since the optimal parameters for ablation, such as power and duration, vary largely due to sizeable intra-patient differences in thickness of the local heart wall, perfusion, blood pressure, etc., it is important to be able to monitor the progress of the lesion development in the tissue to prevent damages to the patient caused by underheating or overheating of the ablation site.

US 2006/0030844 A1 discloses an ablation catheter having an optical sensing unit for monitoring the progress of treatment, which is arranged such that a sensing direction is aligned with the longitudinal axis of the ablation catheter. The sensing characteristics, in particular, the monitoring of lesion development, depend on the orientation of the ablation catheter with respect to the tissue surface. For example, the sensing characteristics are optimal when the ablation catheter is facing the surface of the tissue in substantially a right angle. For other orientations of the ablation catheter with respect to the tissue surface the sensing characteristics are reduced. In particular, for certain orientations of the ablation catheter with respect to the surface of the tissue, the sensing unit might not even be able to sense the contact site of the tissue, at which the ablation catheter contacts the tissue. Thus, the sensing characteristics depend on the orientation of the ablation catheter with respect to the surface of the tissue, thereby reducing the reliability of sensing the object to which energy is applied, in particular, of monitoring of lesion development during an ablation procedure.

### SUMMARY OF THE INVENTION

It is an object of the present invention to provide a catheter, an apparatus, a method and a computer program for applying energy to an object, which allow improving the reliability of sensing the object to which energy is applied.

In an aspect of the present invention a catheter for applying energy to an object is presented, the catheter having a longitudinal axis, wherein the catheter includes:

an energy applying unit for applying energy to the object and

a sensing unit for sensing the object in a sensing direction, wherein the sensing unit is adapted to be rotatable with respect to the catheter such that the angle between the sensing direction of the sensing unit and the longitudinal axis of the catheter is adjustable.

The invention is based on the idea that by providing a sensing unit that is adapted to be rotatable with respect to the catheter it is possible to adjust the sensing unit during the application of energy to an object such that the sensing direction of the sensing unit points to the site to which the energy is applied, regardless of the angle that the catheter tip forms with the surface of the object at that site. This makes it possible to monitor the effect of the energy application even if the orientation of the catheter tip with respect to the object surface changes during the procedure, thereby improving the reliability of sensing the object to which energy is applied.

The term “sensing direction” herein denotes the direction in which the sensing unit is adapted to sense its surroundings. In particular, if the sensing unit is adapted to sense its surroundings within a certain angular range, that is, the sensing unit has a certain opening or sensing angle, the term “sensing direction” may denote a mean or central direction of the angular range.

The energy applying unit applies energy to the object. This energy is, for example, optical or electrical energy. Furthermore, the energy applying unit can have a low temperature for denaturizing tissue by coldness, in particular, by cryoablation, if the object is tissue of a patient, for example, heart tissue of a patient. Thus, the apparatus for applying energy to an object, in particular, the energy applying unit, is preferentially used for an ablation procedure performed at the heart of a patient.

It is preferred that the catheter comprises an expandable catheter region and that the sensing unit is rotatably arranged in the expandable catheter region. This makes it possible to also accommodate longer sensing units, which, due to their length, could not be rotatably arranged within the original diameter of the catheter. For example, the length of a sensing unit comprising an ultrasound transducer with backing material is determined to a large part by the dimension of the backing material behind the transducer. A typical length of this backing material is 4 mm for a transducer diameter of 2 mm. By arranging such a long sensing unit in an expandable catheter region, the sensing unit can be given the necessary space to rotate. Moreover, the use of an expandable catheter region has the further advantage that it allows to provide an unexpanded configuration of the catheter, that is, a configuration in which the diameter of the expandable catheter region is substantially equal to the original diameter of the catheter. In this configuration, the catheter can easily be guided to the site of the object to which energy is to be applied. Moreover, if the object to which energy is applied is tissue of a patient, for example, heart tissue of a patient, the rotatable arrangement of the sensing unit in the expandable catheter region, that is, separated from the atrial blood pool, enhances the security of the patient, because if the transducer were allowed to move freely inside the atrium, this could cause mechanical damage to the heart and/or expose internal (potentially thrombogenic) components of the catheter to the blood.

The expandable catheter region is preferentially or comprises preferentially an expandable balloon and could also be regarded as a balloon section of the catheter.

It is further preferred that a wall thickness of the expandable catheter region is adapted at different positions to predetermine an expanded shape of the expandable catheter region. This allows predetermining the expanded shape of the expandable catheter region such that it is well adapted to the particular rotatable arrangement of the sensing unit. For example, if the sensing unit is rotatable around an axis of rotation arranged in a center region of the sensing unit, such that the sensing unit describes—during rotation—substantially a circular trajectory, the wall thickness of the expandable catheter region can be adapted such that its expanded shape matches this circular trajectory where it protrudes out of the original diameter of the catheter.

Preferentially, the catheter comprises a first condition in which the expandable catheter region is unexpanded and the sensing direction of the sensing unit is aligned with the longitudinal axis of the catheter and a second condition in which the expandable catheter region is expanded to give space for the sensing unit to rotate, wherein the catheter is adapted to be changeable between the first condition and the second condition. Providing a first condition in which the expandable catheter region is unexpanded and the sensing unit is aligned with the longitudinal axis of the catheter makes it possible to easily guide the catheter to the site of the object to which energy is to be applied. In particular, if the object is tissue of a patient, for example, heart tissue of a patient, and the catheter for applying energy to an object, in particular, the energy applying unit, is used for an ablation procedure performed at the heart of a patient, the first, unexpanded condition allows for a simple guidance of the catheter through the veins to the ablation site. Since the sensing direction of the sensing unit is aligned with the longitudinal axis of the catheter in this condition, that is, it is forward-looking, the sensing unit can be used to monitor the guidance of the catheter. Once the catheter has been guided to the site of the object to which energy is to be applied, the catheter can be changed into the second, expanded condition to allow for the rotation of the sensing unit.

It is also preferred that the catheter comprises at least two pull-wires being operationally connected to the sensing unit for rotating the sensing unit. The term “operationally connected” may hereby particularly denote a direct connection between the pull-wires and the sensing unit or an indirect connection, wherein the pull-wires are connected to the sensing unit only indirectly via additional elements, such as mechanical force converter, etc. The use of such pull-wires provides a simple and robust way for rotating the sensing unit. Alternatively, a local motor control, provided in the catheter, can be used for rotating the sensing unit.

It is further preferred that the sensing unit comprises a front end and a rear end, the front end extending into the sensing direction and the rear end extending into a direction opposite to the sensing direction, and that the sensing unit is rotatable around an axis of rotation that is closer to the front end than to the rear end of the sensing unit. Preferentially, the axis of rotation is close to the front end of the sensing unit, for example, it is preferred that the distance between the axis of rotation and the front end is less than 30 percent, even more preferred, less than 10 percent, of the total length (from front end to rear end) of the sensing unit. By rotating the sensing unit around an axis of rotation that is closer to the front end than to the rear end of the sensing unit and, preferentially, close to the front end, the rear end of the sensing unit will protrude—during rotation—farther out of the original diameter of the catheter than the front end. This is particularly advantageous when the catheter tip is to form a substantially sharp angle with the surface of the object to which energy is applied, because it allows providing an expandable catheter region with an expanded shape that is less expanded close to the catheter tip, where the front end of the sensing unit is located, and more expanded farther away from the catheter tip, where the back end of the sensing unit is located. The use of an expandable catheter section with such an “asymmetric” expanded shape then allows for an orientation of the catheter at a sharper angle with respect to the surface of the object to which energy is applied.

Preferentially, the expandable catheter region is expandable by means of an expansion fluid introducible into the catheter. This expansion fluid can be, for example, a gas, or, preferentially, a liquid. By using an expansion fluid for expanding the expandable catheter region, further mechanics, such as additional wires, can be avoided in the catheter.

It is preferred that the catheter further includes an irrigation unit for irrigating the object to which energy is applied with an irrigation fluid, wherein the irrigation fluid and the expansion fluid are the same. Using the same fluid both for irrigating the object and for expanding the expandable catheter region allows for a simpler design of the catheter without requiring supply mechanisms for a plurality of different fluids.

The expansion fluid and/or the irrigation fluid are or comprise preferentially a saline or similar solution.

Preferentially, the sensing unit comprises an ultrasound transducer, which is, for example, a single ultrasound transducer or an ultrasound transducer array. Ultrasound is a well established technology for sensing objects, in particular, if the object is tissue of a patient, for example, heart tissue of a patient, it is well suited for sensing a property of the heart tissue such as the progress of a lesion development during an ablation procedure, in vivo. Alternatively, the sensing unit can also use another imaging modality, for example, the sensing unit be based on an optical imaging with visible or other suitable light.

It is preferred that the sensing unit further comprises a fluid lens, wherein the ultrasound transducer and the fluid lens are rotatable as a unit. By using an ultrasound transducer with a fluid lens, a sensing unit with a relatively large sensing angle can be provided. For example, the opening or sensing angle of a typical fluid lens is about 50 degrees. By rotating the ultrasound transducer and the fluid lens together as a unit, only a comparably small rotation range of the sensing unit, for example, a rotation range of about 35 degrees, is required to cover a large field of view. This smaller rotation range can make the design of the inventive catheter more robust.

It is further preferred that the energy applying unit comprises an acoustically transparent electrode for applying energy to the object. The use of an energy applying unit comprising an acoustically transparent electrode, for example, a TPX electrode with a thin metallic coating on the frontside or an electrode made of an acoustically transparent conductive material such as a doped polymer, makes it possible to use a sensing unit comprising an ultrasound transducer for sensing the object. Such an acoustically transparent electrode is disclosed, for example, in US 2006/0030844 A1, which is herewith incorporated by reference.

In a further aspect of the present invention a method for applying energy to an object is presented, wherein the method comprises the following steps:

applying energy to the object by means of an energy applying unit included in a catheter, the catheter having a longitudinal axis, and

sensing the object in a sensing direction by means of a sensing unit included in the catheter,

wherein the sensing unit is rotated with respect to the catheter such that the angle between the sensing direction of the sensing unit and the longitudinal axis of the catheter is adjusted.

In a further aspect of the present invention an apparatus for applying energy to an object is presented, wherein the apparatus comprises:

a catheter as defined in claim 1 and

a sensing control unit being operationally connectable to the sensing unit of the catheter for controlling the rotation of the sensing unit.

The term “operationally connectable” may hereby particularly denote any kind of connection of the sensing control unit and the sensing unit of the catheter that allows controlling the rotation of the sensing unit. For example, if the catheter comprises at least two pull-wires for rotating the sensing unit, the sensing control unit can be operationally connected to the sensing unit via the pull-wires. If a local motor control, provided in the catheter, is used for rotating the sensing unit, the sensing control unit can be operationally connected to the sensing unit, for example, by wire or wireless.

In a further aspect of the present invention a computer program for applying energy to an object is presented, wherein the computer program comprises program code means for causing a computer to carry out the steps of the method as defined in claim 12, when the computer program is run on a computer controlling an apparatus as defined in claim 13.

It shall be understood that the catheter of claim 1, the method of claim 12, the apparatus of claim 13 and the computer program of claim 14 have similar and/or identical preferred embodiments, in particular, as defined in the dependent claims.

It shall be understood that preferred embodiments of the invention can also be any combination of the dependent claims with a respective independent claim.

### BRIEF DESCRIPTION OF THE DRAWINGS

These and other aspects of the invention will be apparent from and elucidated with reference to the embodiments described hereinafter. In the following drawings

FIG. 1 shows schematically and exemplarily an embodiment of an apparatus for applying energy to an object,

FIG. 2 shows schematically and exemplarily an embodiment of a sensing unit for use in a catheter,

FIG. 3A, 3B show schematically and exemplarily an embodiment of a distal end of a catheter for applying energy to an object in a first and second condition,

FIG. 3C shows schematically and exemplarily another embodiment of a distal end of a catheter for applying energy to an object in a second condition,

FIG. 4 shows schematically and exemplarily a cross section of an embodiment of a catheter for applying energy to an object and

FIG. 5 illustrates schematically and exemplarily an embodiment of a method for applying energy to an object.

### DETAILED DESCRIPTION OF EMBODIMENTS

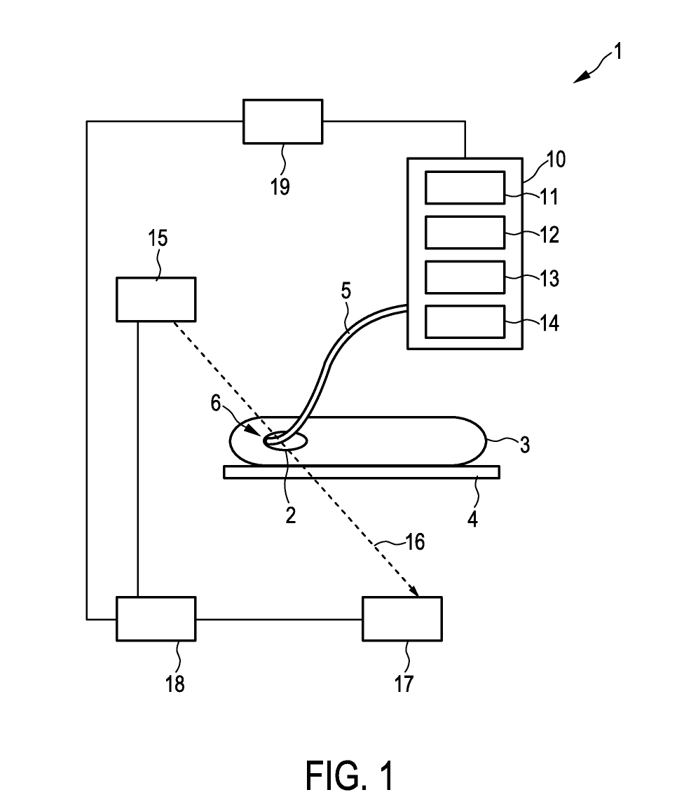

FIG. 1 shows schematically and exemplarily an apparatus 1 for applying energy to an object 2. The object 2 is, in this embodiment, a heart 2 of a patient 3, who is located on a patient table 4. The apparatus 1 comprises a catheter 5 including an energy applying unit for applying energy to the object 2 and a sensing unit for sensing the object 2 in a sensing direction. In this embodiment, the energy applying unit and the sensing unit are located at a distal end 6 of the catheter 5. The distal end 6 of the catheter 5 including the energy applying unit and the sensing unit will be described in more detail further below.

The apparatus 1 for applying energy to an object 2 further comprises a catheter control unit 10, which comprises an electrical energy source 11, in particular, a radio frequency source, for applying electrical energy, in particular, radio frequency energy, to the object 2 via the energy applying unit of the catheter 5.

The catheter control unit 10 further comprises a guiding control unit 12 for guiding the distal end 6 of the catheter 5 to a desired location within the object 2. The guiding control unit 12, in this embodiment, controls build-in guiding means (not shown in FIG. 1) of the catheter 5. In another embodiment, the catheter 5 can, for example, also be steered and navigated by the use of steering-wires in order to passively guide the distal end 6 of the catheter 5 to the desired location within the object 2. Also, the steering-wires can be controlled by the guiding control unit 12.

During the guidance of the distal end 6 of the catheter 5 to the desired location within the object 2 and preferentially also during the application of energy to the object 2 a fluoroscopy device images the location of the distal end 6 of the catheter 5 within the patient 3 and, in particular, within the heart 2, which in this embodiment is the object 2. The fluoroscopy device comprises an X-ray source 15, which generates an X-ray beam 16, which is schematically shown in FIG. 1, for traversing a region of the patient 3, in which the catheter 5, in particular, the distal end 6 of the catheter 5, is present. After the X-ray beam 16 has traversed the patient 3, the X-ray beam 16 is detected by an X-ray detector 17. The X-ray source 15 and the X-ray detector 17 are controlled by a fluoroscopy control unit 18, which preferentially also comprises a display for showing a fluoroscopy image.

In another embodiment, instead of a fluoroscopy device, another imaging device, for example, a magnetic resonance imaging device, an ultrasonic imaging device or a computed tomography imaging device, can be used for imaging the location of the distal end 6 of the catheter 5 within the patient 3 and, in particular, within the heart 2.

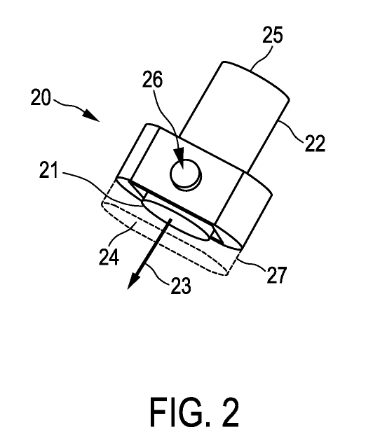

FIG. 2 shows schematically and exemplarily a sensing unit 20 for use in a catheter 5 for applying energy to an object 2. Similar reference signs in FIGS. 1 and 2 denote similar elements.

In the embodiment shown in FIG. 2, the sensing unit 20 is an acoustic sensing unit for acoustically sensing the object 2. The sensing unit 20 comprises an ultrasound transducer 21 with backing material 22 behind the transducer. The backing material 22 is adapted to attenuate acoustic pulses generated by the ultrasound transducer 21 at the back side of the transducer. Such an ultrasound transducer 21 with backing material 22 is known, for example, from U.S. Pat. No. 4,382,201 A1, which is herewith incorporated by reference. In another embodiment, the sensing unit 20 can comprise a thin ultrasound transducer 21, for example, a thin capacitive micro-machined ultrasound transducer (cMUT) or a thin piezo-electric micro-machined ultrasound transducer (pMUT), without backing material 22. In yet another embodiment, the sensing unit 20 can also use another imaging modality, for example, the sensing unit 20 can be an optical sensing unit for optically sensing the object 2 with visible or other suitable light.

In this embodiment, the sensing unit 20 further comprises a fluid lens 27 and the ultrasound transducer 21 and the fluid lens 27 are rotatable as a unit. In other embodiments, the sensing unit 20 can be an acoustic, optical or other sensing unit without a fluid lens 27.

The sensing unit 20 is adapted to sense the object 2 in a sensing direction 23. The sensing unit 20 comprises a front end 24 extending into the sensing direction 23 and a rear end 25 extending into a direction opposite to the sensing direction 23. In the embodiment shown in FIG. 2, the sensing unit 20 is rotatable around an axis of rotation 26 that is closer to the front end 24 than to the rear end 25 of the sensing unit 20. In other embodiments, the axis of rotation 26 can also be in a center of the sensing unit 20 or even closer to the rear end 25 than to the front end 24 of the sensing unit.

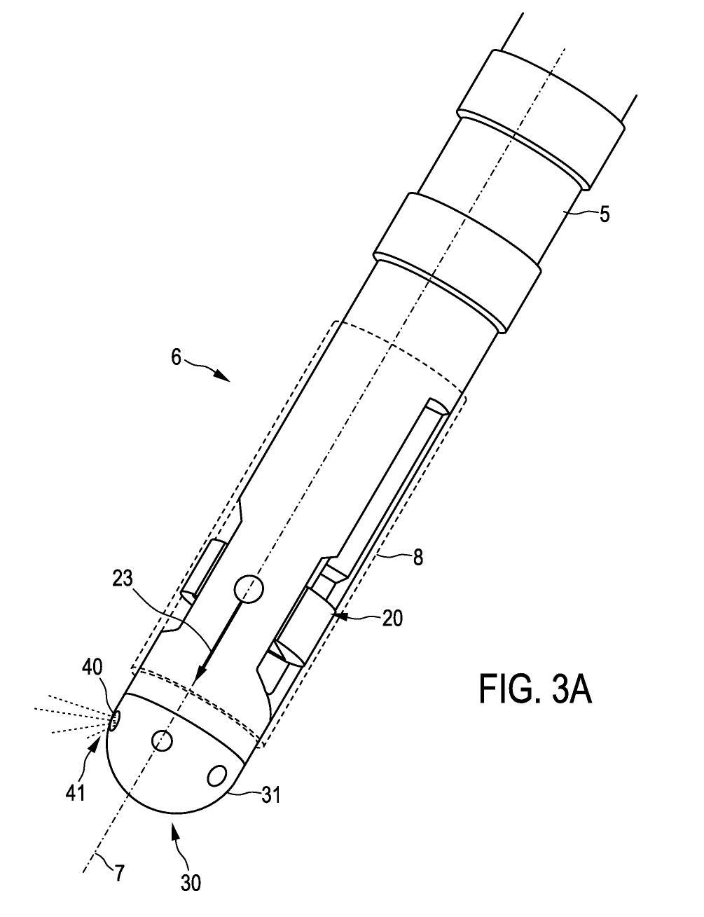

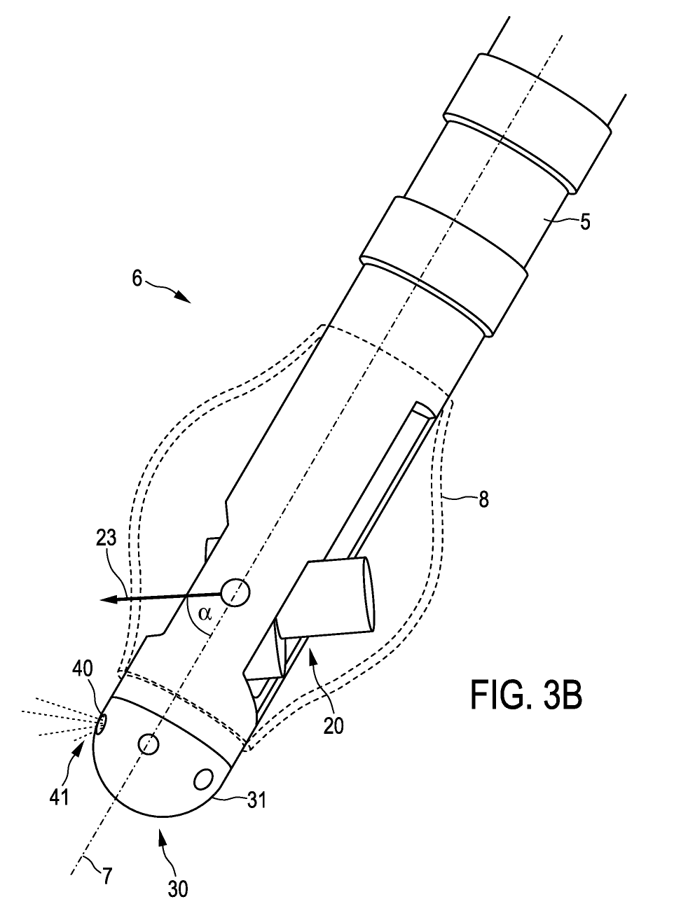

FIGS. 3A and 3B show schematically and exemplarily an embodiment of a distal end 6 of a catheter 5 for applying energy to an object 2 in a first and second condition. Similar reference signs in FIGS. 1, 2, 3A and 3B denote similar elements.

The catheter 5 comprises a longitudinal axis 7 and includes a sensing unit 20 for sensing the object 2 in a sensing direction 23. In this embodiment, the sensing unit 20 is an acoustic sensing unit for acoustically sensing the object 2. In another embodiment, the sensing unit 20 can also use another imaging modality, for example, the sensing unit 20 can be an optical sensing unit for optically sensing the object 2 with visible or other suitable light.

The sensing unit 20 is adapted to be rotatable with respect to the catheter 5 such that the angle α between the sensing direction 23 of the sensing unit 20 and the longitudinal axis 7 of the catheter 5 is adjustable. In the embodiment shown in FIGS. 3A and 3B, the catheter 5 comprises an expandable catheter region 8 and the sensing unit 20 is rotatably arranged in the expandable catheter region 8. The wall of the expandable region 8 is transparent for the sensing unit 8, allowing the sensing unit to view through the region. Optionally, only the part of the wall that comes in the direct view of the sensing unit when this is rotated is made transparent. If the sensing unit is an acoustically sensing unit, the expandable region 8, or the relevant part of it, is made acoustically transparent.

The expandable catheter region 8 is, in this embodiment, a balloon section of the catheter 5.

In the first condition of the catheter 5, shown in FIG. 3A, the expandable catheter region 8 is unexpanded and the sensing direction 23 of the sensing unit 20 is aligned with the longitudinal axis 7 of the catheter 5. In this condition, the angle α between the sensing direction 23 of the sensing unit 20 and the longitudinal axis 7 of the catheter 5 is substantially equal to zero.

In the second condition of the catheter 5, shown in FIG. 3B, the expandable catheter region 8 is expanded to give space for the sensing unit 20 to rotate. In this condition, the angle α between the sensing direction 23 of the sensing unit 20 and the longitudinal axis 7 of the catheter 5 is adjustable. The catheter 5, in this embodiment, is adapted to be changeable between the first condition and the second condition.

In other embodiments, in particular if the length of the sensing unit 20 is short enough to be rotatably arranged within the original diameter of the catheter 5, the catheter 5 does not have to comprise an expandable catheter region 8. In this case, the catheter 5 does not comprise different first and second conditions but rather only a single condition in which the sensing unit 20 is rotatable.

The catheter 5 further includes an energy applying unit 30 for applying energy to the object 2. The energy applying unit 30 comprises a catheter electrode 31, in particular, in this embodiment, a radio frequency catheter electrode 31, which is connectable to the electrical energy source 11 via a contact lead (not shown in FIGS. 3A and 3B). Thus, electrical energy, in this embodiment, radio frequency energy, can be applied to the object 2 via the energy applying unit 30 of the catheter 5.

The catheter electrode 31 of the energy applying unit 30 is, in this embodiment, an acoustically transparent electrode 31 for applying energy to the object 2.

In the embodiment shown in FIGS. 3A and 3B, a wall thickness of the expandable catheter region 8 is adapted at different positions to predetermine an expanded shape of the expandable catheter region 8.

In this embodiment, the catheter 5 further comprises an irrigation unit 40 for irrigating the object 2 to which energy is applied with an irrigation fluid 41. The irrigation unit 40 preferentially comprises at least one hole or nozzle or other opening through which the irrigation fluid 41 for irrigating the object 2 can be sprinkled. For example, the irrigation unit 40 can comprise a single opening or multiple openings. In FIGS. 3A to 3C the irrigation fluid 41 is shown flowing through one opening only for clarity reasons. However, the irrigation fluid can flow through several openings or only through one opening.

Preferentially, the flow of irrigation fluid 41 through the irrigation unit 40 is adjustable, for example, by completely or partly closing the irrigation unit 40, that is, by completely or partly closing the at least one hole or nozzle.

The expandable catheter region 8, in this embodiment, is expandable by means of an expansion fluid introducible into the catheter 5. Thus, the catheter 5 can be changed from the first, unexpanded condition to the second, expanded condition by introducing the expansion fluid into the catheter 5. Likewise, the catheter 5 can be changed from the second, expanded condition to the first unexpanded condition by removing, in particular partly removing the expansion fluid from the catheter 5. In this embodiment, the irrigation fluid 41 is also used as the expansion fluid for expanding the expandable catheter region 8.

Referring back to FIG. 1, the catheter control unit 10 further comprises, in this embodiment, an irrigation control unit 13 for controlling the irrigation unit 40 of the catheter 5. Preferentially, the irrigation control unit 13 comprises a pump, which is connectable to the irrigation unit 40 of the catheter 5, for example, via a tube (not shown in FIG. 1) within the catheter 5. The irrigation control unit 13 is adapted to introduce an irrigation fluid 41 into the catheter 5 which, in this embodiment, is also used as the expansion fluid for expanding the expandable catheter region 8. The joint irrigation/expansion fluid 41, preferentially a saline or similar solution, is thus used, in this embodiment, both for expanding the expandable catheter region 8 and for irrigating the object 2 via the irrigation unit 40. Preferentially, the flow of the joint irrigation/expansion fluid 41 through the irrigation unit 40 is adjusted during the expansion of the expandable catheter region 8. In other embodiments, the catheter 5 does not have to comprise an irrigation unit 40. In this case, the expansion of the expandable catheter region 8 by means of an expansion fluid may be controlled by a suitable expansion control unit which corresponds to the irrigation control unit 13.

The catheter control unit 10 further comprises a sensing control unit 14 for controlling the rotation of the sensing unit 20 and the sensing of the object 2 by the sensing unit 20. In this embodiment, the catheter 5 comprises pull-wires (not shown in FIG. 1) for rotating the sensing unit 20 and the sensing control unit 14 is operationally connected to the pull-wires and controls the rotation of the sensing unit 20 via the pull-wires. In another embodiment, a local motor control, provided in the catheter 5, can be used for rotating the sensing unit 20 and the sensing control unit 14 can be operationally connected to the sensing unit 20, for example, by wire or wireless. Preferentially, the sensing control unit 14 also comprises a display for showing the sensed information of the object 2. In this embodiment, the sensing unit 20 is an acoustic sensing unit for acoustically sensing the object 2 and the sensed information comprises one or more images generated from ultrasound, which is emitted by the ultrasound transducer 21 of the sensing unit 20 and which is reflected by the object 2. In another embodiment, the sensing unit 20 can be, for example, an optical sensing unit for optically sensing the object 2 and the sensed information can comprise one or more images generated from visible or other suitable light.

Preferentially, the sensing unit 20 is adapted for measuring the angle that the catheter tip, that is, the tip of the distal end 6 of the catheter 5, forms with the surface of the object 2 to which energy is applied. In this embodiment, this is preferentially achieved by determining the distance between the sensing unit 20 and the surface of the object 2 in the sensing direction 23 from the ultrasound which is emitted by the ultrasound transducer 21 of the sensing unit 20 and which is reflected by the object 2. The connecting line between the axis of rotation 26 of the sensing unit 20 and the surface of the object 2 in the sensing direction 23 and the connecting line between the axis of rotation 26 of the sensing unit 20 and the surface of the object 2 in the direction of the longitudinal axis 7 of the catheter 5 then define two sides of a triangle with the angle between these two sides being the angle α. From the determined distance between the sensing unit 20 and the surface of the object 2 in the sensing direction 23 and the known distance between the sensing unit 20 and the catheter tip, the angle between the catheter tip and the surface of the object 2 can then be determined.

Preferentially, the sensing unit 20 is further adapted to allow for a sweep movement, wherein the sensing control unit 13 controls the sensing unit 20 such that the angle α between the sensing direction 23 of the sensing unit 20 and the longitudinal axis 7 of the catheter 5 the sensing unit 20 is continuously adjusted between two limiting angles [αₘᵢₙ, αₘₐₓ] in order to provide a larger field of view. These limiting angles [αₘᵢₙ, αₘₐₓ] may be manually selectable by a user, for example, via a suitable user interface provided by the sensing control unit 14 (not shown in FIG. 1), or they may be automatically determined, for example, in dependence of the determined angle between the catheter tip and the surface of the object 2. Alternatively or additionally, the limiting angles [αₘᵢₙ, αₘₐₓ] may also be determined in dependence of the size of the local region of the object surface to which energy is applied.

The apparatus 1 for applying energy to an object 2 further comprises an apparatus control unit 19 for controlling the apparatus 1 for applying energy to an object 2, in particular, for controlling the catheter control unit 10 and preferentially the fluoroscopy or other imaging device. In particular, the apparatus control unit 19 preferentially controls the electrical energy source 11, the guiding control unit 12, the irrigation control unit 13 and the sensing control unit 14.

FIG. 3C shows schematically and exemplarily another embodiment of a distal end 6 of a catheter 5 for applying energy to an object 2 in a second condition. Similar reference signs in FIGS. 1, 2, 3A, 3B and 3C denote similar elements.

In this embodiment, the sensing unit 20 is rotatable around an axis of rotation 26 that is closer to the front end 24 than to the rear end 25 of the sensing unit 20 and a wall thickness of the expandable catheter region 8 is adapted at different positions to predetermine an expanded shape of the expandable catheter region 8 that is less expanded close to the catheter tip, that is, the tip of the distal end 6 of the catheter 5, where the front end 24 of the sensing unit 20 is located, and more expanded farther away from the catheter tip, where the back end 25 of the sensing unit 20 is located. Moreover, in this embodiment, the expandable catheter region 8 is adapted such that during rotation of the sensing unit 20 only that side of the expandable catheter region 8 at which the sensing unit 20 protrudes out of the original diameter of the catheter 5, is expanded.

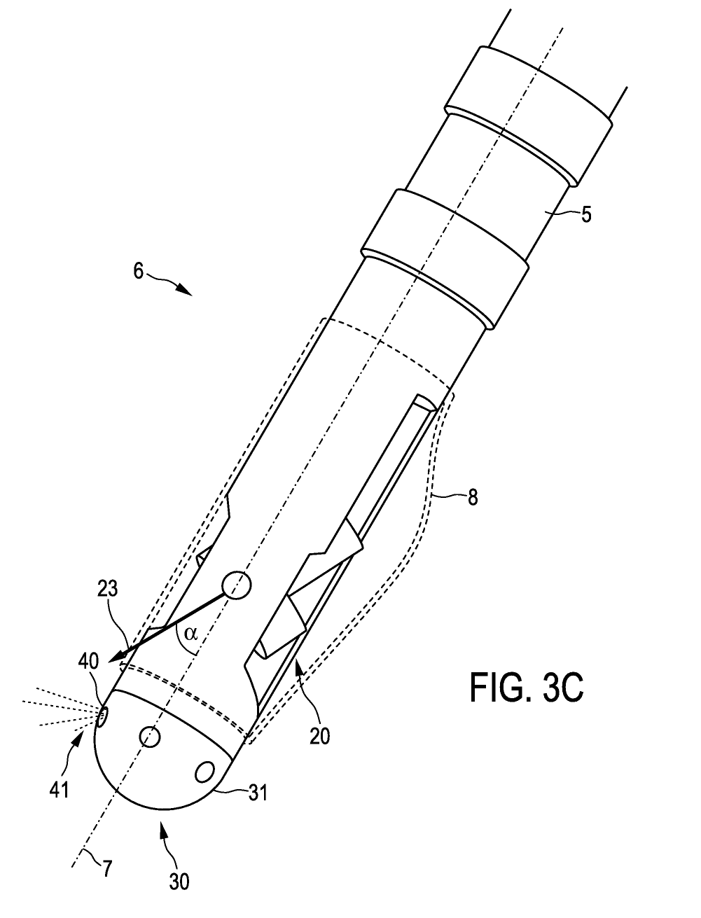

FIG. 4 shows schematically and exemplarily a cross section of an embodiment of a catheter 5 for applying energy to an object 2. Similar reference signs in FIGS. 1, 2, 3A, 3B, 3C and 4 denote similar elements.

The catheter 5 comprises, in this embodiment, a steering-wire 50 and a metal bending strip 52, which are used to steer and navigate the distal end 6 of the catheter 5 to the described location within the object 2. The steering-wire 50 and preferentially the metal bending strip 52 are connectable to the guiding control unit 12, which controls the guiding of the distal end 6 of the catheter 5. The metal bending strip 52 is further furnished with electrical wires or contact leads 54 for, for example, connecting the energy applying unit 30 comprising the catheter electrode 31, in particular, in this embodiment, the radio frequency catheter electrode 31, to the electrical energy source 11. The catheter 5 further comprises pull-wires 51, in this embodiment, two pull-wires 51, which are operationally connected to the sensing unit 20 for rotating the sensing unit 10. The pull-wires 51 are operationally connectable to the sensing control unit 14, which controls the rotation of the sensing unit 20 and the sensing of the object 2 by the sensing unit 20. Furthermore, the catheter 5 comprises a tube 53, which is connected, in this embodiment, to the irrigation unit 40 and to the expandable catheter region 8. The tube 53 is connectable to the irrigation control unit 13, which controls the irrigation of the object 2. Preferentially, the irrigation control unit 13 can also be controlled by the sensing control unit 14 in order to control the expansion of the expandable catheter region 8.

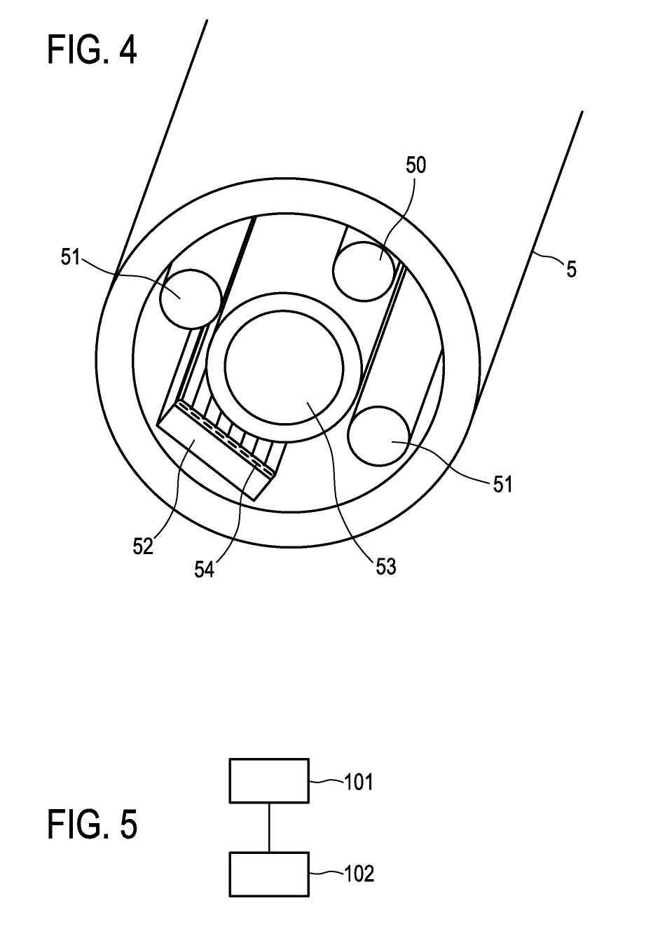

In the following, an embodiment of a method for applying energy to an object 2 will be described with reference to FIG. 5 with respect to the embodiment of a distal end 6 of a catheter 5 for applying energy to an object 2 shown in FIGS. 3A and 3B or 3C. Similar reference signs in FIGS. 1, 2, 3A, 3B, 3C, 4 and 5 denote similar elements.

It is assumed that the distal end 6 of the catheter 5 has been guided to a desired location within the patient 3 and, in particular, within the heart 2, which in this embodiment is the object 2, and that the distal end 6 of the catheter 5 including the energy applying unit 30 is in close proximity, in particular, in contact, with the object 2.

During the guidance of the distal end 6 of the catheter 5, the catheter 5 has been, in this embodiment, in a first condition in which the expandable catheter region 8 is unexpanded and the sensing direction 23 of the sensing unit 20 is aligned with the longitudinal axis 7 of the catheter 5. Since the sensing direction 23 of the sensing unit 20 is aligned with the longitudinal axis 7 of the catheter 5 in this condition, that is, it is forward-looking, the sensing unit 20 has preferentially been used to monitor the guidance of the catheter.

Before the application of energy to the heart 2, the catheter 5 has been changed from the first condition to a second condition in which the expandable catheter region 8 is expanded to give space for the sensing unit 20 to rotate.

In other embodiments, in particular if the length of the sensing unit 20 is short enough to be rotatably arranged within the original diameter of the catheter 5, the catheter 5 does not have to comprise an expandable catheter region 8. In this case, the catheter 5 does not comprise different first and second conditions but rather only a single condition in which the sensing unit 20 is rotatable.

In step 101 of the method for applying energy to an object 2, energy is applied to the heart 2 by the energy applying unit 30, that is, by the catheter electrode 31, in particular, in this embodiment, the radio frequency catheter electrode 31, which is connected to the electrical energy source 11 via a contact lead.

In step 102, which can be performed simultaneously with step 101, the heart 2 is sensed by the sensing unit 20, which, in this embodiment, is an acoustic sensing unit. The sensing unit 20 is rotated with respect to the catheter 5 such that the angle α between the sensing direction 23 of the sensing unit 20 and the longitudinal axis 7 of the catheter 5 is adjusted.

The rotation of the sensing unit 20 with respect to the catheter 5 makes it possible to monitor the effect of the energy application even if the orientation of the catheter tip, that is, the tip of the distal end 6 of the catheter 5, with respect to the surface of the object 2 changes during the procedure, thereby improving the reliability of sensing the object 2 to which energy is applied.

Although in the above described embodiments, the object is preferentially a heart of a patient, in other embodiments, energy can be applied to other objects, for example, to other organs of the patient. Furthermore, the apparatus for applying energy to an object can also be applied to technical objects, in particular, to hollow technical objects, wherein energy has to be applied within these objects.

Although in the above described embodiments, the expansion fluid and the irrigation fluid are the same, the catheter can also comprise at least two separate fluids, that is, at least one expansion fluid and at least one irrigation fluid. In this case, the catheter comprises preferentially at least two tubes, wherein at least one of the tube guides the expansion fluid into the expandable catheter region for expanding the expandable catheter region and wherein at least one other tube of the at least two tubes guides the irrigation fluid to openings for allowing the fluid to leave the catheter for irrigation.

Instead of or in addition to the irrigation fluid, a fluid fulfilling another function can be used. For example, a cooling fluid can be used for cooling the catheter, wherein the cooling fluid preferentially does not leave the catheter. Also the cooling fluid and the expansion fluid can be the same. Furthermore, the irrigation fluid can also have the function to cool the catheter.

While the invention has been illustrated and described in detail in the drawings and foregoing description, such illustration and description are to be considered illustrative or exemplary and not restrictive; the invention is not limited to the disclosed embodiments. Other variations of the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single device or other unit may fulfill the functions of several items recited in the claims. There mere fact that certain measures are recited in mutually different dependent claims does not indicate that a combination of these measures cannot be used to advantage.

Several functions which are realized by different units in the above-described embodiments can be realized by any number of units, also by a single unit.

A computer program may be stored/distributed on a suitable medium, such as an optical storage medium or a solid-state medium supplied together with or as part of other hardware, but may also be distributed in other forms, such as via the internet or other wired or wireless telecommunication systems.

Any reference signs in the claims should not be construed as limiting the scope of the invention.

1. A catheter for applying energy to an object (2), the catheter (5) having a longitudinal axis (7), wherein the catheter (5) includes:
an energy applying unit (30) for applying energy to the object (2) and
a sensing unit (20) for sensing the object (2) in a sensing direction (23), wherein the sensing unit (20) is adapted to be rotatable with respect to the catheter (5) such that the angle (α) between the sensing direction (23) of the sensing unit (20) and the longitudinal axis (7) of the catheter (5) is adjustable.

2. The catheter as defined in claim 1, wherein the catheter (5) comprises an expandable catheter region (8) and wherein the sensing unit (20) is rotatably arranged in the expandable catheter region (8).

3. The catheter as defined in claim 2, wherein a wall thickness of the expandable catheter region (8) is adapted at different positions to predetermine an expanded shape of the expandable catheter region (8).

4. The catheter as defined in claim 2, wherein the catheter (5) comprises a first condition in which the expandable catheter region (8) is unexpanded and the sensing direction (23) of the sensing unit (20) is aligned with the longitudinal axis (7) of the catheter (5) and a second condition in which the expandable catheter region (8) is expanded to give space for the sensing unit (20) to rotate, wherein the catheter (5) is adapted to be changeable between the first condition and the second condition.

5. The catheter as defined in claim 1, wherein the catheter (5) comprises at least two pull-wires (51) being operationally connected to the sensing unit (20) for rotating the sensing unit (20).

6. The catheter as defined in claim 1, wherein the sensing unit (20) comprises a front end (24) and a rear end (25), the front end (24) extending into the sensing direction (23) and the rear end (25) extending into a direction opposite to the sensing direction (23), and wherein the sensing unit (20) is rotatable around an axis of rotation (26) that is closer to the front end (24) than to the rear end (25) of the sensing unit (20).

7. The catheter as defined in claim 1, wherein the expandable catheter region (8) is expandable by means of an expansion fluid (41) introducible into the catheter (5).

8. The catheter as defined in claim 7, wherein the catheter (5) further includes an irrigation unit (40) for irrigating the object (2) to which energy is applied with an irrigation fluid (41) and wherein the irrigation fluid and the expansion fluid are the same.

9. The catheter as defined in claim 1, wherein the sensing unit (20) comprises an ultrasound transducer (21).

10. The catheter as defined in claim 9, wherein the sensing unit (20) further comprises a fluid lens (27) and wherein the ultrasound transducer (21) and the fluid lens (27) are rotatable as a unit.

11. The catheter as defined in claim 1, wherein the energy applying unit (30) comprises an acoustically transparent electrode (31) for applying energy to the object (2).

12. A method for applying energy to an object (2), wherein the method comprises the following steps:
applying energy to the object (2) by means of an energy applying unit (eau) included in a catheter (5), the catheter (5) having a longitudinal axis (7), and
sensing the object (2) in a sensing direction (23) by means of a sensing unit (20) included in the catheter (5),
wherein the sensing unit (20) is rotated with respect to the catheter (5) such that the angle (α) between the sensing direction (23) of the sensing unit (20) and the longitudinal axis (7) of the catheter (5) is adjusted.

13. An apparatus for applying energy to an object (2), wherein the apparatus comprises:
a catheter (5) as defined in claim 1 and
a sensing control unit (14) being operationally connectable to the sensing unit (20) of the catheter (5) for controlling the rotation of the sensing unit (20).

14. A computer program for applying energy to an object (2), wherein the computer program comprises program code means for causing a computer to carry out the steps of the method as defined in claim 12, when the computer program is run on a computer controlling an apparatus as defined in claim 13.
